(19) United States
(12) Patent Application Publication
(10) Pub. No.: US 2025/0087297 A1
(43) Pub. Date: Mar. 13, 2025

(54) SYSTEM AND METHOD FOR ELECTROSTATICS-PRECONDITIONED DRUG MOLECULE GENERATION AND INTERACTION VISUALIZATION

(71) Applicant: QuanMol Tech, Inc., Hillsborough, CA (US)

(72) Inventors: Bo Li, San Carlos, VA (US); Jie Li, San Carlos, CA (US); Minzhen Yi, Hillsborough, CA (US); Jieyu Lu, San Carlos, CA (US); Xudong Lv, Pasadena, CA (US); Xingyu Shen, San Carlos, CA (US)

(21) Appl. No.: 18/243,922

(22) Filed: Sep. 8, 2023

(51) Int. Cl. G16B 15/30; G06F 30/25; G16B 15/20; G16B 40/00

(52) U.S. Cl. CPC G16B 15/30; G06F 30/25; G16B 15/20; G16B 40/00

(57) ABSTRACT

This disclosure presents a method and system aimed at facilitating drug development using electrostatics-based preconditioning. An example method may include computing electrostatic potentials of atoms in a protein molecule. Also, the method may include obtaining a drug molecule for binding to the protein molecule. Furthermore, the method may include modifying (i.e., preconditioning) the drug molecule based on the electrostatic potentials of the atoms in the protein molecule, where the modifying may include: placing charged functional groups on the drug molecule based on absolute values of the electrostatic potentials of the atoms in the protein molecule; and placing polar functional groups or heteroatoms into the drug molecule based on relative values of the electrostatic potentials of the atoms in the protein molecule.

### TECHNICAL FIELD

The disclosure relates generally to preconditioning drug molecules based on electrostatics of atoms in protein for drug generation, as well as visualizing the electrostatic potentials to facilitate the drug molecule preconditioning.

### BACKGROUND

Electrostatic forces play a crucial role in anchoring and binding drug molecules. In general, proteins establish a distinct electrostatic potential distribution, which directly impacts the electrostatic compatibility with drug molecules and, consequently, the strength of molecular binding. However, as of now, there are no effective methods to accomplish the following two tasks: (1) efficiently designing or generating molecules based on the electrostatic potential distribution using a set of algorithms and (2) offering an intuitive visualization that enables medicinal chemists to grasp the electrostatic distribution within a binding site, thus aiding them in determining where to place polar functional groups. This disclosure presents a unified foundational logic and achieves the aforementioned objectives through an algorithmic framework.

### SUMMARY

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, a computer-implemented method may include computing electrostatic potentials of atoms in a protein molecule. The computer-implemented method may also include obtaining a drug molecule for binding to the protein molecule. Method may furthermore include modifying the drug molecule based on the electrostatic potentials of the atoms in the protein molecule, where the modifying may include: placing charged functional groups on the drug molecule based on absolute values of the electrostatic potentials of the atoms in the protein molecule; and placing polar functional groups or heteroatoms into the drug molecule based on relative values of the electrostatic potentials of the atoms in the protein molecule. Other embodiments of this aspect include corresponding computer systems, apparatus, and computer programs recorded on one or more computer storage devices, each configured to perform the actions of the methods.

In some embodiment, the computing electrostatic potentials of atoms in the protein molecule may include: computing an electrostatic potential for each atom based on a position of the atom within the protein molecule. In some embodiment, the computing electrostatic potentials of atoms in the protein molecule may include: computing an electrostatic potential for a Gaussian distribution surrounding each atom in the protein molecule; and using the electrostatic potential for the Gaussian distribution surrounding the atom as the electrostatic potential for the atom.

In some embodiment, the placing charged functional groups on the drug molecule based on absolute values of the electrostatic potentials of the atoms in the protein molecule may include: if the absolute value of the electrostatic potential of an atom is negative, identifying a binding site in the drug molecule corresponding to the atom, and adding a functional group having an electric charge of +1 or a higher positive integer charges to the binding site of the drug molecule.

In some embodiment, the placing charged functional groups on the drug molecule based on absolute values of the electrostatic potentials of the atoms in the protein molecule may include: if the absolute value of the electrostatic potential of an atom is positive, identifying a binding site in the drug molecule corresponding to the atom, and adding a functional group having an electric charge of −1 or a higher negative integer charges to the binding site of the drug molecule.

In some embodiment, the placing polar functional groups or heteroatoms into the drug molecule based on relative values of the electrostatic potentials of the atoms in the protein molecule may include: identifying a region in the protein molecule that is more negatively charged based on the relative values of the electrostatic potentials of atoms in the region; and placing one or more heteroatoms to a binding site in the drug molecule that corresponds to the region in the protein molecule.

In some embodiment, a total number of charges in the drug protein stays the same after the placing polar functional groups or heteroatoms into the drug molecule based on relative values of the electrostatic potentials of the atoms in the protein molecule.

In some embodiments, the computer-implemented method may further include: constructing a molecule fragment library from a compound database, where the molecule fragment library may include structure patterns exist in the compound database; restraining the modifying of the drug molecule based on the molecule fragment such that a modified molecule fragment is required to exist in the molecule fragment library.

In some embodiment, the constructing the molecule fragment library may include: constructing the molecule fragment library as a graph data structure, where each node in the graph data structure represents a functional group of atoms, neighboring nodes of a given node represent permissible modified variants of the given node according to the compound library, and edges between nodes represent modification operators.

In some embodiment, the modifying the drug molecule may include: identifying a molecule fragment in the drug molecule that is to be modified; searching for a first node in the graph data structure that corresponds to the molecule fragment; iteratively searching for an edge associated with the first node that represents a desired modification operator; and in response to the edge being found, obtaining a second node associated with the edge as a modified version of the molecule fragment.

In some embodiments, the method may further include: visualizing the electrostatic potentials of atoms in the protein molecule by using point clouds surrounding the atoms, wherein: (1) radiuses of the point clouds are greater than radiuses of the atoms; and (2) point clouds corresponding to atoms with positive electrostatic potentials are visualized in a first color, and point clouds corresponding to atoms with negative electrostatic potentials are visualized in a second color.

In some embodiment, the modification operators may include: placing a charged functional group, a polar functional group, or a heteroatom. Implementations of the described techniques may include hardware, a method or process, or a computer tangible medium.

In one general aspect, a system may include one or more processors, and a memory storing instructions that, when executed by the one or more processors, cause the system to: compute electrostatic potentials of atoms in a protein molecule; obtain a drug molecule for binding to the protein molecule; modify the drug molecule based on the electrostatic potentials of the atoms in the protein molecule, wherein the modifying comprises: place charged functional groups on the drug molecule based on absolute values of the electrostatic potentials of the atoms in the protein molecule; and place polar functional groups or heteroatoms into the drug molecule based on relative values of the electrostatic potentials of the atoms in the protein molecule.

In one general aspect, a non-transitory computer-readable medium may include one or more instructions that, when executed by one or more processors of a device, cause the device to: compute electrostatic potentials of atoms in a protein molecule, obtain a drug molecule for binding to the protein molecule, and modify the drug molecule based on the electrostatic potentials of the atoms in the protein molecule, where the modifying may include: placing charged functional groups on the drug molecule based on absolute values of the electrostatic potentials of the atoms in the protein molecule; and placing polar functional groups or heteroatoms into the drug molecule based on relative values of the electrostatic potentials of the atoms in the protein molecule.

These and other features of the systems, methods, and non-transitory computer-readable media disclosed herein, as well as the methods of operation and functions of the related elements of structure and the combination of parts and economics of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for purposes of illustration and description only and are not intended as a definition of the limits of the invention. It is to be understood that the foregoing general description and the following detailed description are exemplary and explanatory only, and are not restrictive of the invention, as claimed.

### BRIEF DESCRIPTION OF THE DRAWINGS

Preferred and non-limiting embodiments of the invention may be more readily understood by referring to the accompanying drawings in which:

FIG. 1 illustrates an example system diagram for electrostatics-preconditioned drug molecule generation, in accordance with various embodiments.

FIG. 2 illustrates an example method for electrostatics-preconditioned drug molecule generation, in accordance with various embodiments.

FIG. 3 illustrates an example screenshot of the visualization of electrostatic potentials of atoms in a protein structure for electrostatics-preconditioned drug molecule generation, in accordance with various embodiments.

FIG. 4 illustrates a block diagram of an example computer system in which any of the embodiments described herein may be implemented.

### DETAILED DESCRIPTION OF THE EMBODIMENTS

Specific, non-limiting embodiments of the present invention will now be described with reference to the drawings. It should be understood that particular features and aspects of any embodiment disclosed herein may be used and/or combined with particular features and aspects of any other embodiment disclosed herein. It should also be understood that such embodiments are by way of example and are merely illustrative of a small number of embodiments within the scope of the present invention. Various changes and modifications obvious to one skilled in the art to which the present invention pertains are deemed to be within the spirit, scope, and contemplation of the present invention as further defined in the appended claims.

In order to grasp the technical advantages of the solution described here, it is important to have an understanding of the prevailing approach in the field of drug discovery, molecular design, molecular simulation, computational chemistry, and partially, machine learning and geometric deep learning.

A commonly employed method for drug candidate generation involves using sizable collections of chemical compounds known as compound libraries. These libraries usually include a large collection of diverse chemical compounds that can be screened to identify potential drug candidates. In the traditional drug discovery process, the first step is to identify a specific target, such as a protein or receptor, that is associated with a particular disease or condition. Once a specific target is identified, the next step involves selecting a suitable compound library. The choice of the library depends on several factors, such as the target's characteristics, the desired mechanism of action, the chemical diversity needed to explore different molecular interactions, and the commercial availability or synthetic feasibility of the compounds. These considerations are sometimes coupled with structural patterns of previous drugs for related protein targets (e.g., conventional kinase inhibitors are characterized by having a planar hinge-binding group with dual or more H-bonding sites). An iterative screening process is then performed to test a large number of compounds against the target. This process is generally lengthy and inefficient.

During the iterative screening, various computational or experimental techniques are employed to evaluate the compounds based on specific criteria. One important aspect of this process is the use of scoring functions. In general, compound scoring can be obtained from approximate static structure-based models (e.g., Vina, GLIDE, TankBind, IGN) or more comprehensive molecular simulation-based protocols (e.g., free-energy perturbation (FEP)) that estimate the affinity or likelihood of a compound binding to the target (e.g., a protein receptor) or possessing certain properties.

The scoring functions are designed to predict the binding affinity, stability, or other relevant properties of the compounds. These functions consider factors such as molecular shape, electrostatic interactions, hydrogen bonding, hydrophobicity, and other physicochemical properties. The compounds are typically ranked or scored based on their predicted activity or fitness for the target.

However, despite the extensive use of scoring functions, it is challenging to develop a scoring function that is universally effective and accurate for all scenarios. The effectiveness of a scoring function depends on the specific target, the chemical properties of the compounds, and the nature of the interactions involved. Achieving high accuracy in predicting compound activities or properties remains a significant challenge in the field.

In summary, generating compound libraries and relying on scoring functions for iterative screening can be a slow process, and there is no scoring function that is considered sufficiently effective in all cases. Consequently, the success rate of virtual screening remains at a low level of typically 5-10%.

To address these technical challenges, the method and system described herein compute and utilize electrostatic potentials of atoms in a protein molecule to guide molecular modification/growth (e.g., electrostatics-preconditioning) of a drug molecule. By considering the electrostatic properties of a protein's active site, drug designers can strategically position charged or polar functional groups on drug molecules, enhancing binding affinity, specificity, and efficacy. In essence, these potentials guide the rational design of drugs that form strong, targeted interactions with protein targets, increasing the likelihood of successful drug-protein interactions in therapeutic applications. The system can guide the modification or growth of drug molecules in a more targeted and streamlined manner. In addition, this approach avoids the need for the extensive generation of compound libraries and the subsequent iterative screening process.

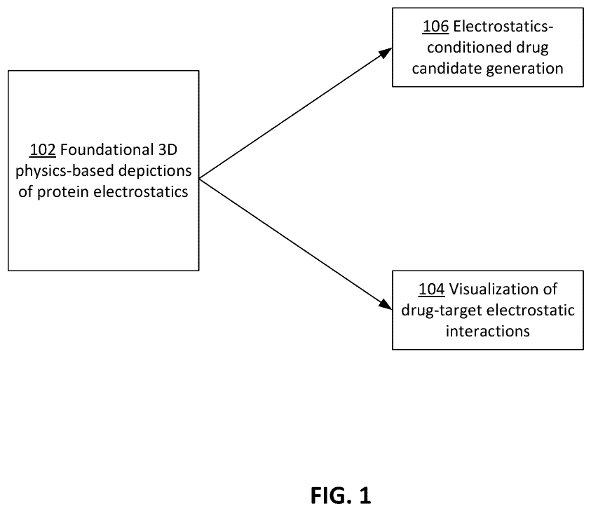

FIG. 1 illustrates an example system diagram for electrostatics-preconditioned drug molecule generation, in accordance with various embodiments. In practical terms, the electrostatic potential distribution in the protein dictates the optimal positioning of “polar functional groups” and “heteroatoms” within drug molecules, providing invaluable guidance for drug design. The multiple components in FIG. 1 are for illustrative purposes. Depending on the implementation, the system may include more, fewer, or alternative components.

In some embodiments, the system in FIG. 1 may include a backend module 102 for computing protein electrostatics and the 3D physics-based depictions of the per-atom electrostatic potentials, a first downstream module 104 for generating visualization of drug-target electrostatics interactions, and a second downstream module 106 for guiding electrostatics-conditioned drug candidate modification and generation.

The backend module 102 can be implemented on a server device or a cloud service, while the downstream modules 104 and 106 may include user-interfaces that interact with researchers. The user-interfaces can take the form of either a desktop application or a web-based application. For example, the user-interface might involve a desktop application installed directly on a user's computer or a web-based application accessed through a web browser. The back-end module 102 is responsible for sending computed data to the desktop application or the web-based application via internet connections. This allows the desktop application or the web-based application to generate visualizations of drug-target shape matching and/or provide guidance in the modification and generation of drug candidates.

In some embodiments, the backend module 102 computes per-atom electrostatic potentials for the atoms in the protein. The electrostatic potential of an atom in a protein refers to the electric potential energy experienced by a charged particle (e.g., the atom) due to the presence of other charged particles within the protein and its surrounding environment.

There are various ways for the backend module 102 to compute the per-atom electrostatic potentials (ESP). The first way is to compute ESP for each atom solely based on the atom's position, which is straightforward, less computation-intensive, but could lack information about the microenvironment that can be explored by the atom's thermal motions. The second way is to compute ESP for each atom based on a Gaussian distribution surrounding the atom, which is more computationally expensive but provides a more comprehensive characterization of the atom's surrounding environment. In some other embodiments, analogous atom-centered three-dimensional point sampling may also be devised to obtain similar results.

Calculating the electrostatic potential of an atom within a molecule entails determining the electric potential at a precise location in space, taking into account the influence of all other charges present in the molecule. Whether the Electrostatic Potential (ESP) is computed solely based on the atom's position or considers the Gaussian distribution around the atom, the ESP calculation can be carried out by the backend module 102 using either a high-precision electrostatic model or semi-empirical quantum mechanics.

For instance, the computation process may start with obtaining a three-dimensional structure of the protein molecule of interest, e.g., from X-ray crystallography or NMR spectroscopy, or from protein structure databases. The process may be followed by extracting the atomic coordinates of the protein and surrounding solvent molecules from the protein structure. In one embodiment, one of various high-precision electrostatic models may be selected to perform the electrostatic calculations based on the atomic coordinates of the atoms, such as Poisson-Boltzmann models, finite-difference methods, or boundary element methods.

In another embodiment in which semi-empirical quantum mechanics are used for computing the ESP, a specialized quantum chemistry software package like Gaussian, xTB, GAMESS, or MOPAC may be used to approximate the computational environment. The atomic coordinates of the protein may then be input into the chosen semi-empirical quantum mechanics software to obtain the ESPs. Notably, the effectiveness of the current approach is conditioned on an accurate description of the pocket's electrostatic landscape, which prevents the use of traditional fixed-charge force field models that are widely employed in biomolecular simulations. Instead, at least a high-accuracy polarizable electrostatic model or a more sophisticated quantum-mechanical approach must be employed.

When the ESP computation is based on the Gaussian distribution surrounding the atoms, it may involve solving the Poisson's equation to model the electrostatic interactions between charged particles. Note that the Gaussian distribution is used to approximate the electron density around atoms. In some embodiments, the first step is to model the electron density around the atoms using a Gaussian distribution. This involves determining parameters like the position, width, and amplitude of the Gaussian function that represents the electron density. Subsequently, a Poisson equation may be constructed to describe the relationship between the electrostatic potential and the electron density and charges. Solving Poisson's equation involves discretizing the Gaussian distribution space into a grid and calculating the potential at each grid point. Common methods include Finite Difference methods or solving the Poisson-Boltzmann equation for biomolecular systems. Other electrostatic models might be solved differently depending on the model details, for instance: (i) The coarse-grained electrostatic model (C-GeM) involves minimization of an approximate coarse-grained electronic Hamiltonian to determine the charge distribution and hence the electrostatics. (ii) Charge-equilibration models (QEq) require matrix diagonalization to solve an approximate linear system for partial charges, which can be further polarized using Newton-Raphson methods (e.g., as in the polarizable QEq or PQEq method). Some embodiments are built upon a combination of multiple such methods in order to establish a consensus of foundational electrostatic descriptions, which increase the confidence of subsequent application modules.

In some embodiments, when the ESP computation is based on the Gaussian distribution surrounding the atoms, hydrogen (H) atoms may be specially treated. For instance, instead of directly calculating a spherical Gaussian distribution centered on the H atom, the computation may be based on a semi-spherical Gaussian distribution that is centered on the H atom but is positioned away from the other atom (e.g., a carbon atom (C) in a C—H bond). In other words, the semi-spherical Gaussian distribution is deliberately skewed or biased away from the other bonded atom. This bias ensures that the distribution is primarily focused on one side of the H atom, away from its neighboring atom. This is because hydrogen is lighter and smaller than essentially all other atoms, which makes C—H bonds appear differently compared to C—C bonds. In particular, the distance between C—H is smaller than the distance between C—C. If the bond is C—F (Fluorine), the distance between C and F would also be greater than the distance between C—H. Therefore, the ESP computation based on “the semi-spherical Gaussian distribution centered on the H atom but away from the other atom” biases the calculations towards the more distant portion of H. This makes it more relevant for performing atomic substitutions from H to heavier atoms.

Once the backend module 102 computes the per-atom electrostatic potentials for the atoms, it may generate point cloud objects for all or some of the atoms in the protein or the target molecule based on the electrostatic potentials. For example, the electrostatic potentials may first go through a quantization process and be converted into a plurality of quantized values. The quantization process is designed to discretize or represent the continuous values of the electrostatic potentials in a discrete form. This quantization process aids in establishing machine-readable interaction encodings that can be subsequently used in machine-learning-based preconditioning options.

In some embodiments, the downstream module 104 may generate a visualization of the drug-target electrostatic interactions based on the point cloud objects corresponding to the per-atom electrostatic potentials. In some embodiments, the point cloud objects may be color-coded, using a first color representing positive electrostatic potentials and a second color representing negative positive electrostatic potentials. These color codes greatly facilitate researchers or users to identify the desired modification of the corresponding sites on the drug molecule. For instance, if the color of a point cloud surrounding an atom indicates a positive electrostatic potential, the researcher may trigger a chemical reaction to introduce negatively charged elements or ions to the corresponding binding site on the drug molecule.

In some embodiments, the shape or size of the point cloud surrounding an atom also indicate the strength of the electrostatic potential of the atom. That is, the overall shape of a pocket on the protein molecule (formed by the shapes of the point clouds surrounding the atoms) is not about the structural shape of the protein (e.g., the boundaries of the protein), but rather the electrostatic-potential of the pocket on the protein.

In some embodiments, the downstream module 106 may perform pre-conditioning or modifications to the drug molecule based on the electrostatic potentials of the atoms in the protein molecule to guide the drug candidate generation. The electrostatic potentials of the atoms in the protein molecule may provide precise information for placing complementary charges (+1 and −1) on the drug molecule. This allows the drug molecule to interact favorably with these regions on the protein molecule through electrostatic attractions. The electrostatic interactions can significantly contribute to the binding affinity between molecules and play a crucial role in anchoring the bound molecule at a specific conformation. By introducing charged groups on the drug molecule that align with oppositely charged regions on the protein, it creates additional attractive forces that can enhance the stability of the drug-protein complex. In addition, the electrostatic potentials of the atoms provide information about the spatial distribution of charges on the protein surface. By designing the charged groups on the drug molecule to align with specific regions of the protein, it increases the likelihood of a specific and targeted interaction. Other technical benefits may include guided docking and optimization, structural fit and conformational adaptation, tailored interactions and drug properties, etc.

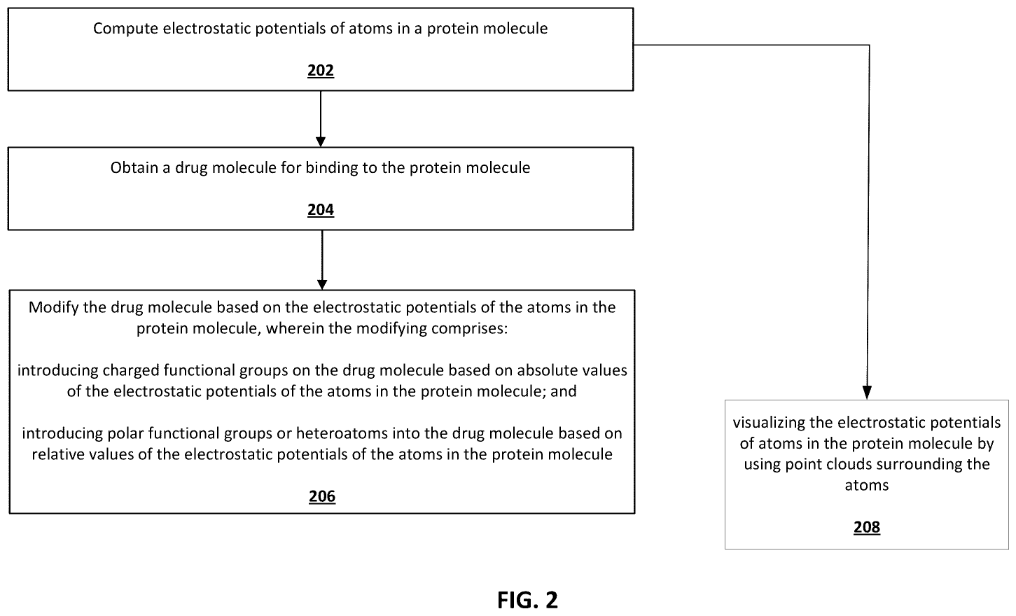

Another significant advantage of this approach is to incorporate prior conditions (the electrostatics potentials of the atoms represented as point clouds) without the need for post-generation screening. In other words, this point-cloud representation-based solution circumvents the traditional reliance on compound library screening by directly generating new molecules based on shape pre-conditioning. FIG. 2 provides more detailed embodiments implementing the downstream modules 104 and 106.

FIG. 2 illustrates an example method for electrostatics-preconditioned drug molecule generation, in accordance with various embodiments. In some implementations, one or more process blocks of FIG. 2 may be performed by a device.

As shown in FIG. 2, process 200 may include computing electrostatic potentials of atoms in a protein molecule (block 202). For example, device may compute electrostatic potentials of atoms in a protein molecule, as described in FIG. 1. Process 200 may further include obtaining a drug molecule for binding to the protein molecule (block 204) and modifying the drug molecule based on the electrostatic potentials of the atoms in the protein molecule (block 206).

In some embodiments, the modification in block 206 may include one or more chemical modifications such as case (1): introducing charged functional groups on the drug molecule based on absolute values of the electrostatic potentials of the atoms in the protein molecule; and case (2): introducing polar functional groups or heteroatoms into the drug molecule based on relative values of the electrostatic potentials of the atoms in the protein molecule. For drug-like structures, the above-identified case (1) typically corresponds to ammonium, guanidinium, imidazolium, carboxylate, sulfonate, phosphorate, etc., case (2) referring to halogen, carbonyl, hydroxyl, amide, nitrile, as well as other possible backbone heteroatom replacements, especially those in ring structures. These structures can be introduced at various sites of the molecule to increase bioactivity and drug likeliness.

In other words, the absolute values of electrostatic potentials are used to guide the binding between a pocket on the protein molecule with a charged functional group. In some embodiments, the charged functional groups specifically refer to groups carrying a +1/−1 or higher integer charge. For instance, if a pocket has an overall positive electrostatic potential (based on the per-atom potentials), an electrostatically negatively charged functional group may be introduced to the corresponding binding site on the drug molecule to achieve a more stable binding. If a pocket has an overall negative electrostatic potential (based on the per-atom potentials), an electrostatically negatively charged functional group may be introduced to the corresponding binding site on the drug molecule to achieve a more stable binding. A control of the total charge should additionally account for the mechanism of action as well as the key absorption, distribution, metabolism, excretion and toxicity (ADMET) properties of interest to the current drug-discovery program, which can vary in different embodiments.

In addition, the relative values of electrostatic potentials may be used to improve the binding affinity between a pocket on the protein molecule with a polar functional group or one or more heteroatoms introduced to the drug molecule, in which the total charge remains unchanged after introducing the polar functional group or one or more heteroatoms. As a salient characteristic of these functional groups and heteroatoms, they contribute to drug-target binding through and only through polarization of the partial charge distributions. In some embodiments, heteroatoms are introduced to the drug molecule for binding to regions that is electrostatically negative on the protein molecule, or vice versa. The relative values of electrostatic potentials among the atoms or regions provide a guidance on how to place one or more polar functional groups or heteroatoms to achieve a more stable binding.

Here, functional groups are specific groups of atoms within a molecule that determine its physical, chemical, and biological properties. A charged functional group is one that contains an excess or deficiency of electrons, resulting in a net positive or negative charge. In comparison, a polar functional group here refers to a group of atoms within a molecule that leads to an unequal distribution of electron density, resulting in a partial positive charge on one end and a partial negative charge on the other. This polarity influences the molecule's interactions with other molecules and its solubility in different solvents. At last, atoms like carbon (C) and hydrogen (H) are often considered as the “mainstream” atoms, and heteroatoms are atoms that are not carbon or hydrogen, introducing chemical diversity into the molecule. Common heteroatoms include oxygen (O), nitrogen (N), sulfur(S), and halogens (F, Cl, Br. I). Heteroatoms can significantly affect the chemical behavior and properties of a molecule due to their distinct electronic characteristics and chemical reactivities.

In some embodiments, the drug molecule pre-conditioning or modification may be constrained using a fragment library (also called motif vocabulary). The fragment library may be constructed based on a compound database to ensure that all generated molecular fragments from the preconditioning/modification adhere to the structure patterns already present in the vocabulary. This guarantees the synthesizability of the proposed molecules.

In some embodiments, the fragment library may be represented as a graph data structure, with each node in the graph representing a functional group of atoms, and the neighboring nodes of the node to represent the permissible modified variants of the molecule fragment according to the fragment library. The edges between the molecule fragments represent the modification operators required to realize the transformation. This way, for a given molecule fragment and a desired modification, one can easily look up the graph for the molecule fragment and the desired modification in the associated edges. If both are found, the graph node connected by the edge to the molecule fragment can be obtained to represent the modified variant of the molecule fragment.

Some modification operators may include substitution (substituting one atom or functional group with another), addition (adding new atoms or functional groups to a fragment), deletion (removing atoms or functional groups from a fragment), ring formation or breaking (creating or breaking rings within a fragment), functional group transformation (converting one functional group into another while keeping the rest of fragment intact), rearrangement, oxidation/reduction, etc.

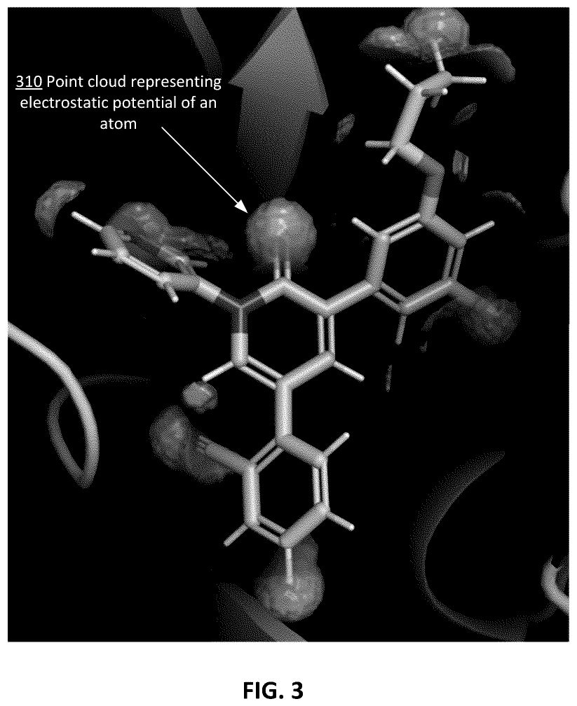

In some embodiments, the visualization of the electrostatic potentials of atoms in the protein molecule may include displaying point clouds surrounding the atoms. The radius of the point cloud surrounding the atom may be greater than the radius of the atom, and proportional to the absolute value of the electrostatic potential. The color of the point cloud may indicate whether the atom has a positive or negative electrostatic potential. FIG. 3 illustrates an example visualization of the electrostatic potentials of atoms (e.g., 310) within a protein (without showing the colors), in which the atoms are enclosed by point clouds showing the electrostatic potentials.

Process 200 may include additional implementations, such as any single implementation or any combination of implementations described below and/or in connection with one or more other processes described elsewhere herein. In a first implementation, the computing electrostatic potentials of atoms in the protein molecule may include: computing an electrostatic potential for each atom based on a position of the atom within the protein molecule.

In a second implementation, alone or in combination with the first implementation, the computing electrostatic potentials of atoms in the protein molecule may include: computing an electrostatic potential for a Gaussian distribution surrounding each atom in the protein molecule; and using the electrostatic potential for the Gaussian distribution surrounding the atom as the electrostatic potential for the atom.

In a third implementation, alone or in combination with the first and second implementation, the placing charged functional groups on the drug molecule based on absolute values of the electrostatic potentials of the atoms in the protein molecule may include: if the absolute value of the electrostatic potential of an atom is negative, identifying a binding site in the drug molecule corresponding to the atom, and adding a functional group having an electric charge of +1 or a higher positive integer charges to the binding site of the drug molecule.

In a fourth implementation, alone or in combination with one or more of the first through third implementations, the placing charged functional groups on the drug molecule based on absolute values of the electrostatic potentials of the atoms in the protein molecule may include: if the absolute value of the electrostatic potential of an atom is positive, identifying a binding site in the drug molecule corresponding to the atom, and adding a functional group having an electric charge of −1 or a higher negative integer charges to the binding site of the drug molecule.

In a fifth implementation, alone or in combination with one or more of the first through fourth implementations, the placing polar functional groups or heteroatoms into the drug molecule based on relative values of the electrostatic potentials of the atoms in the protein molecule may include: identifying a region in the protein molecule that is more negatively charged based on the relative values of the electrostatic potentials of atoms in the region; and placing one or more heteroatoms to a binding site in the drug molecule that corresponds to the region in the protein molecule.

In a sixth implementation, alone or in combination with one or more of the first through fifth implementations, a total number of charges in the drug protein stays the same after the placing polar functional groups or heteroatoms into the drug molecule based on relative values of the electrostatic potentials of the atoms in the protein molecule.

A seventh implementation, alone or in combination with one or more of the first through sixth implementations, process 200 further includes constructing a molecule fragment library from a compound database, where the molecule fragment library may include structure patterns exist in the compound database; restraining the modifying of the drug molecule based on the molecule fragment such that a modified molecule fragment is required to exist in the molecule fragment library.

In an eighth implementation, alone or in combination with one or more of the first through seventh implementations, the constructing the molecule fragment library may include: constructing the molecule fragment library as a graph data structure, where each node in the graph data structure represents a functional group of atoms, neighboring nodes of a given node represent permissible modified variants of the given node according to the compound library. and edges between nodes represent modification operators.

In a ninth implementation, alone or in combination with one or more of the first through eighth implementations, the modifying the drug molecule may include: identifying a molecule fragment in the drug molecule that is to be modified; searching for a first node in the graph data structure that corresponds to the molecule fragment; iteratively searching for an edge associated with the first node that represents a desired modification operator; and in response to the edge being found, obtaining a second node associated with the edge as a modified version of the molecule fragment.

In a tenth implementation, alone or in combination with one or more of the first through ninth implementations, the modification operators may include: placing a charged functional group, a polar functional group, or a heteroatom.

An eleventh implementation, alone or in combination with one or more of the first through tenth implementations, process 200 further includes visualizing the electrostatic potentials of atoms in the protein molecule by using point clouds surrounding the atoms (block 208), where: (1) radiuses of the point clouds are greater than radiuses of the atoms; and (2) point clouds corresponding to atoms with positive electrostatic potentials are visualized in a first color, and point clouds corresponding to atoms with negative electrostatic potentials are visualized in a second color. In some embodiments, the size of a point cloud is proportional to the absolute value of the electrostatic potential of the atom.

With the different sizes and different colors, a user can easily identify and quantify the electrostatic potentials of the atoms or regions in the protein molecule and make guided modification (i.e., preconditioning) to the drug molecule. This approach improves binding affinity (by introducing charged groups on the drug molecule that align with oppositely charged regions on the protein, additional attractive forces can be created to enhance the stability of the drug-protein complex) and specificity (the electrostatic potentials provide information about the spatial distribution of charges on the protein surface. By designing the charged groups on the drug molecule to align with specific regions of the protein, the likelihood of a specific and targeted interaction is increased. This can also reduce off-target interactions and improve the drug's selectivity).

Although FIG. 2 shows example blocks of process 200, in some implementations, process 200 may include additional blocks, fewer blocks, different blocks, or differently arranged blocks than those depicted in FIG. 2. Additionally, or alternatively, two or more of the blocks of process 200 may be performed in parallel.

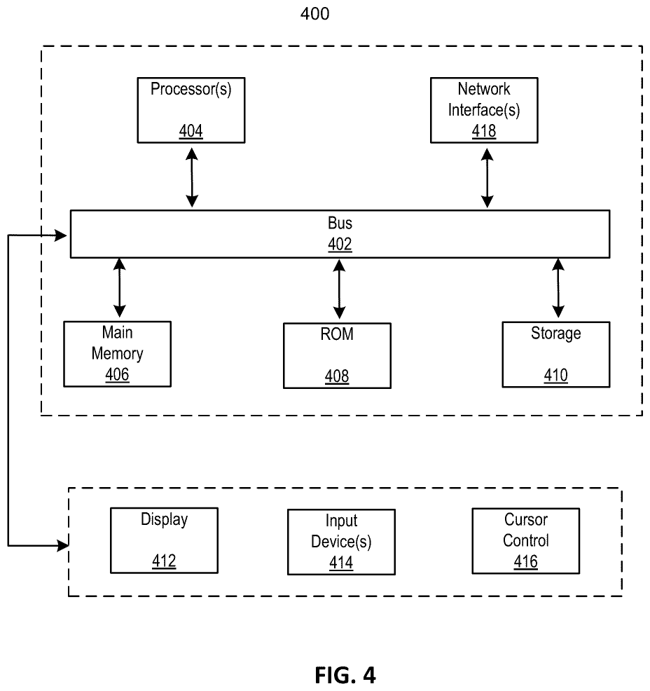

FIG. 4 illustrates a block diagram of an example computer system 400 in which any of the embodiments described herein may be implemented. The computer system 400 includes a bus 402 or other communication mechanisms for communicating information, one or more hardware processors 404 coupled with bus 402 for processing information. Hardware processor(s) 404 may be, for example, one or more general-purpose microprocessors.

The computer system 400 also includes a main memory 406, such as a random access memory (RAM), cache and/or other dynamic storage devices, coupled to bus 402 for storing information and instructions to be executed by processor 404. Main memory 406 also may be used for storing temporary variables or other intermediate information during execution of instructions to be executed by processor 404. Such instructions, when stored in storage media accessible to processor 404, render computer system 400 into a special-purpose machine that is customized to perform the operations specified in the instructions.

The computer system 400 further includes a read-only memory (ROM) 408 or other static storage device coupled to bus 402 for storing static information and instructions for processor 404. A storage device 410, such as a magnetic disk, optical disk, or USB thumb drive (Flash drive), etc., is provided and coupled to bus 402 for storing information and instructions.

The computer system 400 may be coupled via bus 402 to a display 412, such as a cathode ray tube (CRT) or LCD display (or touch screen), for displaying information to a computer user. An input device 414, including alphanumeric and other keys, is coupled to bus 402 for communicating information and command selections to processor 404. Another type of user input device is cursor control 416, such as a mouse, a trackball, or cursor direction keys for communicating direction information and command selections to processor 404 and for controlling cursor movement on display 412. This input device typically has two degrees of freedom in two axes, a first axis (e.g., x) and a second axis (e.g., y), that allows the device to specify positions in a plane. In some embodiments, the same direction information and command selections as cursor control may be implemented via receiving touches on a touch screen without a cursor.

The computing system 400 may include a user interface module to implement a GUI that may be stored in a mass storage device as executable software codes that are executed by the computing device(s). This and other modules may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

In general, the word “module,” as used herein, refers to logic embodied in hardware or firmware, or to a collection of software instructions, possibly having entry and exit points, written in a programming language, such as, for example, Java, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules or computing device functionality described herein are preferably implemented as software modules, but may be represented in hardware or firmware. Generally, the modules described herein refer to logical modules that may be combined with other modules or divided into sub-modules despite their physical organization or storage.

The computer system 400 may implement the techniques described herein using customized hard-wired logic, one or more ASICs or FPGAs, firmware and/or program logic which in combination with the computer system causes or programs computer system 400 to be a special-purpose machine. According to one embodiment, the techniques herein are performed by computer system 400 in response to processor(s) 404 executing one or more sequences of one or more instructions contained in main memory 406. Such instructions may be read into main memory 406 from another storage medium, such as storage device 410. Execution of the sequences of instructions contained in main memory 406 causes processor(s) 404 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 410. Volatile media includes dynamic memory, such as main memory 406. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 402. Transmission media can also take the form of acoustic or light waves, such as those generated during radio-wave and infra-red data communications.

Various forms of media may be involved in carrying one or more sequences of one or more instructions to processor 404 for execution. For example, the instructions may initially be carried on a magnetic disk or solid state drive of a remote computer. The remote computer can load the instructions into its dynamic memory and send the instructions over a telephone line using a modem. A modem local to computer system 400 can receive the data on the telephone line and use an infra-red transmitter to convert the data to an infra-red signal. An infra-red detector can receive the data carried in the infra-red signal and appropriate circuitry can place the data on bus 402. Bus 402 carries the data to main memory 406, from which processor 404 retrieves and executes the instructions. The instructions received by main memory 406 may retrieves and executes the instructions. The instructions received by main memory 406 may optionally be stored on storage device 410 either before or after execution by processor 404.

The computer system 400 also includes a communication interface 418 coupled to bus 402. Communication interface 418 provides a two-way data communication coupling to one or more network links that are connected to one or more local networks. For example, communication interface 418 may be an integrated services digital network (ISDN) card, cable modem, satellite modem, or a modem to provide a data communication connection to a corresponding type of telephone line. As another example, communication interface 418 may be a local area network (LAN) card to provide a data communication connection to a compatible LAN (or WAN component to communicated with a WAN). Wireless links may also be implemented. In any such implementation, communication interface 418 sends and receives electrical, electromagnetic or optical signals that carry digital data streams representing various types of information.

A network link typically provides data communication through one or more networks to other data devices. For example, a network link may provide a connection through local network to a host computer or to data equipment operated by an Internet Service Provider (ISP). The ISP in turn provides data communication services through the world wide packet data communication network now commonly referred to as the “Internet”. Local network and Internet both use electrical, electromagnetic or optical signals that carry digital data streams. The signals through the various networks and the signals on network link and through communication interface 418, which carry the digital data to and from computer system 400, are example forms of transmission media.

The computer system 400 can send messages and receive data, including program code, through the network(s), network link and communication interface 418. In the Internet example, a server might transmit a requested code for an application program through the Internet, the ISP, the local network and the communication interface 418.

The received code may be executed by processor 404 as it is received, and/or stored in storage device 410, or other non-volatile storage for later execution.

Each of the processes, methods, and algorithms described in the preceding sections may be embodied in, and fully or partially automated by, code modules executed by one or more computer systems or computer processors comprising computer hardware. The processes and algorithms may be implemented partially or wholly in application-specific circuitry.

The various features and processes described above may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language, such as, among others, “can,” “could,” “might,” or “may.” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Any process descriptions, elements, or blocks in the flow diagrams described herein and/or depicted in the attached figures should be understood as potentially representing modules. segments, or portions of code which include one or more executable instructions for implementing specific logical functions or steps in the process. Alternate implementations are included within the scope of the embodiments described herein in which elements or functions may be removed, executed out of order from that shown or discussed, including substantially concurrently or in reverse order, depending on the functionality involved, as would be understood by those skilled in the art.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

### Language

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Although an overview of the subject matter has been described with reference to specific example embodiments, various modifications and changes may be made to these embodiments without departing from the broader scope of embodiments of the present disclosure. Such embodiments of the subject matter may be referred to herein, individually or collectively, by the term “invention” merely for convenience and without intending to voluntarily limit the scope of this application to any single disclosure or concept if more than one is, in fact, disclosed.

The embodiments illustrated herein are described in sufficient detail to enable those skilled in the art to practice the teachings disclosed. Other embodiments may be used and derived therefrom, such that structural and logical substitutions and changes may be made without departing from the scope of this disclosure. The Detailed Description, therefore, is not to be taken in a limiting sense, and the scope of various embodiments is defined only by the appended claims, along with the full range of equivalents to which such claims are entitled.

It will be appreciated that an “engine,” “system,” “data store,” and/or “database” may comprise software, hardware, firmware, and/or circuitry. In one example, one or more software programs comprising instructions capable of being executable by a processor may perform one or more of the functions of the engines, data stores, databases, or systems described herein. In another example, circuitry may perform the same or similar functions. Alternative embodiments may comprise more, less, or functionally equivalent engines, systems, data stores, or databases, and still be within the scope of present embodiments. For example, the functionality of the various systems, engines, data stores, and/or databases may be combined or divided differently.

“Open source” software is defined herein to be source code that allows distribution as source code as well as compiled form, with a well-publicized and indexed means of obtaining the source, optionally with a license that allows modifications and derived works.

The data stores described herein may be any suitable structure (e.g., an active database, a relational database, a self-referential database, a table, a matrix, an array, a flat file, a documented-oriented storage system, a non-relational No-SQL system, and the like), and may be cloud-based or otherwise.

As used herein, the term “or” may be construed in either an inclusive or exclusive sense. Moreover, plural instances may be provided for resources, operations, or structures described herein as a single instance. Additionally, boundaries between various resources, operations, engines, engines, and data stores are somewhat arbitrary, and particular operations are illustrated in a context of specific illustrative configurations. Other allocations of functionality are envisioned and may fall within a scope of various embodiments of the present disclosure. In general, structures and functionality presented as separate resources in the example configurations may be implemented as a combined structure or resource. Similarly, structures and functionality presented as a single resource may be implemented as separate resources. These and other variations, modifications, additions, and improvements fall within a scope of embodiments of the present disclosure as represented by the appended claims. The specification and drawings are, accordingly, to be regarded in an illustrative rather than a restrictive sense.

Although the invention has been described in detail for the purpose of illustration based on what is currently considered to be the most practical and preferred implementations, it is to be understood that such detail is solely for that purpose and that the invention is not limited to the disclosed implementations, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment. A component being implemented as another component may be construed as the component being operated in a same or similar manner as the another component, and/or comprising same or similar features, characteristics, and parameters as the another component.

The phrases “at least one of,” “at least one selected from the group of,” or “at least one selected from the group consisting of.” and the like are to be interpreted in the disjunctive (e.g., not to be interpreted as at least one of A and at least one of B).

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment, but may be in some instances. Furthermore, the particular features, structures, or characteristics may be combined in any suitable manner in one or more embodiments.

1. A computer-implemented method, comprising:
computing electrostatic potentials of atoms in a protein molecule;
obtaining a drug molecule for binding to the protein molecule;
modifying the drug molecule based on the electrostatic potentials of the atoms in the protein molecule, wherein the modifying comprises:
introducing charged functional groups on the drug molecule based on absolute values of the electrostatic potentials of the atoms in the protein molecule; and
introducing polar functional groups or heteroatoms into the drug molecule based on relative values of the electrostatic potentials of the atoms in the protein molecule.

2. The computer-implemented method of claim 1, wherein the computing electrostatic potentials of atoms in the protein molecule comprises:
computing an electrostatic potential for each atom based on a position of the atom within the protein molecule.

3. The computer-implemented method of claim 1, wherein the computing electrostatic potentials of atoms in the protein molecule comprises:
determining a Gaussian distribution surrounding each atom in the protein molecule;
computing an electrostatic potential for the Gaussian distribution surrounding each atom in the protein molecule; and
using the electrostatic potential for the Gaussian distribution surrounding the atom as the electrostatic potential for the atom.

4. The computer-implemented method of claim 1, wherein the introducing charged functional groups on the drug molecule based on absolute values of the electrostatic potentials of the atoms in the protein molecule comprises:
if the absolute value of the electrostatic potential of an atom is negative, identifying a binding site in the drug molecule corresponding to the atom, and adding a functional group having an electric charge of +1 or a higher positive integer charges to the binding site of the drug molecule.

5. The computer-implemented method of claim 1, wherein the introducing charged functional groups on the drug molecule based on absolute values of the electrostatic potentials of the atoms in the protein molecule comprises:
if the absolute value of the electrostatic potential of an atom is positive, identifying a binding site in the drug molecule corresponding to the atom, and adding a functional group having an electric charge of −1 or a higher negative integer charges to the binding site of the drug molecule.

6. The computer-implemented method of claim 1, wherein the introducing polar functional groups or heteroatoms into the drug molecule based on relative values of the electrostatic potentials of the atoms in the protein molecule comprises:
identifying a region in the protein molecule that is more negatively charged based on the relative values of the electrostatic potentials of atoms in the region; and
introducing one or more heteroatoms to a binding site in the drug molecule that corresponds to the region in the protein molecule.

7. The computer-implemented method of claim 1, wherein a total number of charges in the drug protein stays the same after the introducing polar functional groups or heteroatoms into the drug molecule based on relative values of the electrostatic potentials of the atoms in the protein molecule.

8. The computer-implemented method of claim 1, further comprising:
constructing a molecule fragment library from a compound database, wherein the molecule fragment library comprises structure patterns exist in the compound database; and
restraining the modifying of the drug molecule based on the molecule fragment such that a modified molecule fragment is required to exist in the molecule fragment library.

9. The computer-implemented method of claim 8, wherein the constructing the molecule fragment library comprises:
constructing the molecule fragment library as a graph data structure, wherein each node in the graph data structure represents a functional group of atoms, neighboring nodes of a given node represent permissible modified variants of the given node according to the compound library, and edges between nodes represent modification operators.

10. The computer-implemented method of claim 9, wherein the modifying the drug molecule comprises:
identifying a molecule fragment in the drug molecule that is to be modified;
searching for a first node in the graph data structure that corresponds to the molecule fragment;
iteratively searching for an edge associated with the first node that represents a desired modification operator; and
in response to the edge being found, obtaining a second node associated with the edge as a modified version of the molecule fragment.

11. The computer-implemented method of claim 9, wherein the modification operators comprise:
introducing a charged functional group, a polar functional group, or a heteroatom.

12. The computer-implemented method of claim 1, further comprising:
visualizing the electrostatic potentials of atoms in the protein molecule by using point clouds surrounding the atoms, wherein:
(1) radiuses of the point clouds are greater than radiuses of the atoms; and
(2) point clouds corresponding to atoms with positive electrostatic potentials are visualized in a first color, and point clouds corresponding to atoms with negative electrostatic potentials are visualized in a second color.

13. A system comprising one or more processors, and a memory storing instructions that, when executed by the one or more processors, cause the system to:
compute electrostatic potentials of atoms in a protein molecule;
obtain a drug molecule for binding to the protein molecule;
modify the drug molecule based on the electrostatic potentials of the atoms in the protein molecule, wherein the modifying comprises:
introduce charged functional groups on the drug molecule based on absolute values of the electrostatic potentials of the atoms in the protein molecule; and
introduce polar functional groups or heteroatoms into the drug molecule based on relative values of the electrostatic potentials of the atoms in the protein molecule.

14. The system of claim 13, wherein to introduce the charged functional groups on the drug molecule based on absolute values of the electrostatic potentials of the atoms in the protein molecule, the instructions cause the system to:
if the absolute value of the electrostatic potential of an atom is negative, identify a binding site in the drug molecule corresponding to the atom, and add a functional group having an electric charge of +1 or a higher positive integer charges to the binding site of the drug molecule; and
if the absolute value of the electrostatic potential of an atom is positive, identify a binding site in the drug molecule corresponding to the atom, and add a functional group having an electric charge of −1 or a higher negative integer charges to the binding site of the drug molecule.

15. The system of claim 13, wherein to introduce the polar functional groups or heteroatoms into the drug molecule based on relative values of the electrostatic potentials of the atoms in the protein molecule, the instructions cause the system to:
identify a region in the protein molecule that is more negatively charged based on the relative values of the electrostatic potentials of atoms in the region; and
introduce one or more heteroatoms to a binding site in the drug molecule that corresponds to the region in the protein molecule.

16. The system of claim 13, wherein the instruction causes the system to further perform:
constructing a molecule fragment library from a compound database, wherein the molecule fragment library comprises structure patterns exist in the compound database; and
restraining the modifying of the drug molecule based on the molecule fragment such that a modified molecule fragment is required to exist in the molecule fragment library.

17. A non-transitory computer-readable medium storing a set of instructions, the set of instructions comprising one or more instructions that, when executed by one or more processors of a device, cause the device to perform operations comprising:
computing electrostatic potentials of atoms in a protein molecule;
obtaining a drug molecule for binding to the protein molecule;
modifying the drug molecule based on the electrostatic potentials of the atoms in the protein molecule, wherein the modifying comprises:
introducing charged functional groups on the drug molecule based on absolute values of the electrostatic potentials of the atoms in the protein molecule; and
introducing polar functional groups or heteroatoms into the drug molecule based on relative values of the electrostatic potentials of the atoms in the protein molecule.

18. The non-transitory computer-readable medium of claim 17, wherein the computing electrostatic potentials of atoms in the protein molecule comprises:
computing an electrostatic potential for each atom based on a position of the atom within the protein molecule.

19. The non-transitory computer-readable medium of claim 17, wherein the computing electrostatic potentials of atoms in the protein molecule comprises:
computing an electrostatic potential for a Gaussian distribution surrounding each atom in the protein molecule; and
using the electrostatic potential for the Gaussian distribution surrounding the atom as the electrostatic potential for the atom.

20. The non-transitory computer-readable medium of claim 17, wherein the introducing charged functional groups on the drug molecule based on absolute values of the electrostatic potentials of the atoms in the protein molecule comprises:
if the absolute value of the electrostatic potential of an atom is negative, identifying a binding site in the drug molecule corresponding to the atom, and adding a functional group having an electric charge of +1 or a higher positive integer charges to the binding site of the drug molecule; and
if the absolute value of the electrostatic potential of an atom is positive, identifying a binding site in the drug molecule corresponding to the atom, and adding a functional group having an electric charge of −1 or a higher negative integer charges to the binding site of the drug molecule.
